(19) United States
(12) Patent Application Publication
(10) Pub. No.: US 2021/0177879 A1
(43) Pub. Date: Jun. 17, 2021

(54) VITAMIN D RECEPTOR AGONIST

(71) Applicant: Guanghzou University of Chinese Medicine, Guangzhou (CN)

(72) Inventor: Shaozhen Hou, Guangzhou (CN)

(21) Appl. No.: 16/609,551

(22) PCT Filed: May 15, 2019
(86) PCT No.: PCT/CN2019/087090

§ 371 Date: Oct. 30, 2019

(30) Foreign Application Priority Data
May 17, 2018 (CN) 201810472547.X

(51) Int. Cl. A61K 31/7048; A61P 35/04

(52) U.S. Cl. CPC A61K 31/7048; A61P 35/04

(57) ABSTRACT

The present invention belongs to the technical field of biomedicine, and in particular relates to a vitamin D receptor agonist, where the agonist includes: one or more of monotropein, desacetyl asperulosidic acid, asperuloside, and asperulosidic acid; and studies have shown that one or more of monotropein, desacetyl asperulosidic acid, asperuloside, and asperulosidic acid can well activate vitamin D receptors, and thus have preventive and therapeutic effects on breast cancer, pancreatic cancer, and colon cancer by activating the vitamin D receptors.

### CROSS-REFERENCE TO RELATED APPLICATIONS

This application is a 371 National Stage Entry of International Application No. PCT/CN2019/087090, filed on May 17, 2019, which claims benefit of Chinese patent application No. 201810472547.X, filed to the National Intellectual Property Administration on May 17, 2018 and entitled “VITAMIN D RECEPTOR AGONIST”, which is herein incorporated by reference in its entirety.

### TECHNICAL FIELD

The present invention belongs to the technical field of biomedicine, and in particular relates to a vitamin D receptor agonist.

### BACKGROUND

A vitamin D is a fat-soluble vitamin that is of great significance for human health, especially for children's health. In addition to causing rickets due to well-known abnormalities in metabolism of calcium and phosphorus and poor calcification, vitamin D deficiency can also affect the functions of various tissues and organs such as nerves, muscles, immunity, and hematopoiesis. After entering the body, the vitamin D is metabolized into an active vitamin D (the final in vivo activated form is a ligand 1,25(OH)₂D₃), which exerts its biological effects by binding to a vitamin D receptor (VDR).

After binding to the ligand, i.e., the active vitamin D, the VDR undergoes a conformational change, which activates the transcription of a target gene and mediates its biological effects through genomic and non-genomic mechanisms. The genomic mechanism activates a second messenger through a nuclear receptor, while the non-genomic mechanism activates the second messenger through a membrane receptor, causing a series of rapid biological effects which are independent of gene transcription and act quickly but not persistently. 1. Immune regulation effects: it has been found that many immune cells in human body, such as monocyte macrophages, activated T lymphocytes and B lymphocytes, all can express VDR, and their immune activities and proliferation and differentiation are all regulated by the active vitamin D. The active vitamin D can preferentially select a helper T1 cell as a target cell, inhibit Th1 proliferation, and inhibit the production of interleukin 2 and interferon gamma; the active vitamin D can also down-regulate the expression of histocompatibility antigen II. Dendritic cells play an important role in cellular immunity. The active vitamin D can inhibit the maturation of dendritic cells, making T cells unable to activate and present antigens, and thus induce immune tolerance. CD4+ T cells can secrete a variety of inhibitors, such as IL-4, IL-10, etc. The active vitamin D can specifically activate CD4+ T cells around a tissue, reduce Th1 proliferation, and inhibit Th1 from secreting IL-2 and IL-5. The active vitamin D can increase Th2 by down-regulating IL-12 expression and up-regulating IL-4 expression, and IL-12 also plays an important role in Th1 differentiation. The active vitamin D can stimulate Th2 to secrete IL-4, IL-5 and IL-10, and transform an immune rejection response towards an immune tolerance response. The active vitamin D can inhibit B lymphocytes from secreting immunoglobulin, and can weaken the immune response of the body by enhancing the function of suppressor T cells. The active vitamin D, after binding to unactivated monocytes, can promote killing of pathogenic microorganisms by cells. The active vitamin D can inhibit the production of T cell growth factors, granulocyte colony-stimulating factors, interleukin and interferon. The effect of the active vitamin D on cellular immunity provides a new theoretical basis for the prevention and treatment of autoimmune diseases, such as diabetes, rheumatoid arthritis, vitiligo, autoimmune thyroiditis, etc. Its role has been confirmed in various animal models. 2. Anti-tumor effects: the anti-tumor mechanism of the VDR and its ligand, the active vitamin D, has the following aspects: (1) blocking cell cycles and inducing cell differentiation. The active vitamin D can block tumor cells in a G1 phase, so that cells of a G0 phase are accumulated, and cells of an S phase are reduced, resulting in decreased expression of cell cycle regulatory proteins and making tumor cell proliferation be decreased. The active vitamin D can increase protein kinase C-induced tumor cell differentiation, and can also induce tumor differentiation by regulating the expression of DNA-binding inhibitors and relying on N-terminal kinases. (2) Inducing apoptosis of the tumor cells. The active vitamin D inhibits tumors primarily by modulating mediators that play a key role in the process of apoptosis. The active vitamin D is closely related to Bcl-2 family genes. Bcl-2 genes are important anti-apoptotic genes in human. The synergistic effect of Bcl family genes and the Bcl-2 genes plays an important role in apoptosis. A large number of studies have shown that the active vitamin D can down-regulate Bcl-2 expression, thereby promoting apoptosis of the tumor cells. (3) Inhibiting metastasis and invasion of tumors. The active vitamin D3 can reduce the expression of serine protein kinases and sulfur proteins, reduce proteolytic enzymes and inhibit its activity, reduce collagenases in cancer cells, reduce the expression of adhesion protein receptors, and inhibit tumor angiogenesis, thereby weakening proliferation, infiltration and transfer of the cancer cells. 3. Effects of regulating calcium and phosphorus metabolism and other effects: the active vitamin D plays its classic role in regulating calcium and phosphorus metabolism as mediated by the VDR, the active vitamin D can promote the synthesis of calcium-binding proteins in small intestinal mucosal cells, increase the absorption of calcium by small intestinal mucosa, and accordingly increase the absorption of phosphorus; increases the reabsorption of calcium and phosphorus, and especially phosphorus by the proximal tubule; directly acts on the mineral metabolism of the bone, promotes the differentiation of osteoclasts, and promotes the proliferation of osteoblasts. The active vitamin D and its analogues can promote the differentiation of peripheral blood mononuclear cells towards macrophages, can up-regulate the expression of insulin-like growth factors I and binding proteins, block the role of the insulin-like growth factors I in promoting cell mitosis, and thus plays an important role in maintaining the physiological functions of normal cells and regulating cell growth.

In view of the above, it is of great importance for doctors and patients to conduct research on new vitamin receptor agonists and get new drugs for clinical use.

### SUMMARY

For the aforementioned reasons, the present invention provides a novel vitamin D receptor agonist including: one or more of monotropein, desacetyl asperulosidic acid, asperuloside, and asperulosidic acid. Studies have shown that one or more of monotropein, desacetyl asperulosidic acid, asperuloside, and asperulosidic acid can well activate vitamin D receptors, and thus have a therapeutic effect on breast cancer, pancreatic cancer, colon cancer and the like by activating the vitamin D receptors.

The present invention is achieved by the following technical solutions.

A vitamin D receptor agonist is disclosed, which includes: one or more of monotropein, desacetyl asperulosidic acid, asperuloside, and asperulosidic acid.

The present invention provides the use of the vitamin D receptor agonist in preparation of a medicament for the treatment and/or prevention of a cancer.

Preferably, the cancer is the colon cancer.

Preferably, the cancer is the breast cancer.

Preferably, the cancer is the pancreatic cancer.

The present invention provides a pharmaceutical preparation prepared from the vitamin D receptor agonist, where the pharmaceutical preparation uses one or more of monotropein, desacetyl asperulosidic acid, asperuloside, and asperulosidic acid as active ingredients.

Preferably, the pharmaceutical preparation includes an oral preparation and an injection preparation.

The present invention provides the use of one or more of monotropein, desacetyl asperulosidic acid, asperuloside, and asperulosidic acid in preparation of a medicament for the treatment and/or prevention of a cancer after the activation of the vitamin D receptors.

The present invention provides the use of the vitamin D receptor agonist for the treatment and/or prevention of a cancer.

Preferably, the cancer includes the colon cancer, the breast cancer and the pancreatic cancer.

### BRIEF DESCRIPTION OF THE DRAWING

FIG. 1 shows a comparison of body weight changes in each group of mice;

FIG. 2 shows the growth of colon tumors in each group of mice;

FIG. 3 shows a comparison of the degree of spleen enlargement in each group of mice;

FIG. 4 is a comparison of the length and thickness of the colon and the number and size of tumors in each group of mice;

FIG. 5 shows a comparison of intestinal permeability in each group of mice;

FIG. 6 shows a comparison of cytokine levels in tumor tissues for each group of mice;

FIG. 7 shows a comparison of the number of macrophages in each group of mice, and a comparison of macrophages M1/M2 in each group of mice;

FIG. 8 shows that the monotropein has no secretory effect on an endogenous VD3 in normal animals;

FIG. 9 shows the level of the endogenous VD3 in each group of mice;

FIG. 10 shows the PGE2 content in the blood and intestine of each group of mice;

FIG. 11 shows the expression of a VDR in the colon for each group of mice;

FIG. 12 shows the expression of VDR in colon cancerous sites of each group of mice;

FIG. 13 shows inhibition of the colon cancer in mice by four compounds such as monotropein;

FIG. 14 shows the colon length of each group of animals;

FIG. 15 shows the colon thickness of each group of animals;

FIG. 16 shows the expression of a VDR protein in the colon for each group of animals;

FIG. 17 shows the expression of the VDR in the colon cancerous sites of each group of animals;

FIG. 18 shows the inhibition of in vitro proliferation of a colon cancer cell line HT29 by four compounds such as the monotropein;

FIG. 19 shows the inhibition of proliferation of a HT29 transplanted tumor by four compounds such as the monotropein;

FIG. 20 shows the inhibition of in vitro proliferation of SW1990 breast cancer cells by four compounds such as the monotropein;

FIG. 21 shows the inhibition of proliferation of a transplanted tumor of SW1990 breast cancer cells by four compounds such as the monotropein;

FIG. 22 shows the inhibition of in vitro proliferation of MB-231 pancreatic cancer cells by four compounds such as the monotropein; and

FIG. 23 shows the inhibition of proliferation of a transplanted tumor of MB-231 pancreatic cancer cells by four compounds such as the monotropein.

### DETAILED DESCRIPTION

The technical solutions of the present invention are described below in connection with specific embodiments, but the claimed scope of the present invention is not limited thereto.

The content described in the embodiments of the specification is merely an enumeration of the implementations of the inventive concept, and the claimed scope of the present invention should not be construed as being limited to the specific forms stated in the embodiments. Equivalent technical means that come into the minds of those of skills in the art in accordance with the inventive concept also fall within the claimed scope of the present invention. Although the embodiments of the present invention are described below, the present invention is not limited to the aforementioned specific embodiments and application fields, and the following specific embodiments are merely illustrative, instructive, rather than restrictive. Those of ordinary skills in the art can make various forms under the inspiration of this specification and without departing from the claimed scope of the claims of the present invention, and these forms are all within the claimed scope of the present invention.

The following experiment of the present invention is a conclusive experiment summarized by the research and development personnel based on the technical solutions to be protected by the present invention on the basis of a plurality of creative experiments.

Experiment 1 Experimental Study on the Anti-Colon Cancer Effect of Monotropein

Experimental method: 90 Balb/c mice of 6-8 weeks were purchased from the Experimental Animal Center in Guangzhou University of Chinese Medicine, weighed about 18-22 g, and subjected to adaptive feeding for 10 days. Modeling was divided into three stages. In the first stage, except for the normal group of animals being given saline injection, each of the other animals were intraperitoneally injected with a carcinogen AOM (10 mg/kg), and given free access to 2% DSS water from the next day for 7 days, and then given access to replaced regular drinking water for 14 days. In the second stage, the animals were given free access to 2% DSS water for 10 days, and then given access to replaced regular drinking water for 14 days. In the third stage, the animals were given free access to 2% DSS water for 10 days, and then given access to replaced regular drinking water for 11 days. The normal group was given access to regular drinking water throughout the experiment. The diet, water drinking and body weights of the animals were recorded daily and DAI scores were made, and the animals were killed and harvested at the 10th week. In this experiment, the animals were divided into groups and administrated on the first day of a modeling period of the second stage, and the total number of groups was set to 5 groups. The first group was a blank control group (Normal), in which a total of 12 animals were given access to drinking water by gavage; the second group was a model group (AOM/DSS), in which a total of 22 animals were given access to drinking water by gavage; the third group was a positive drug control group (5-ASA+AOM/DSS), in which a total of 22 animals were given access to 100 mg/kg of mesalazine (5-ASA) by gavage; and the fourth and fifth groups were monotropein-intervening groups (Mo+AOM/DSS), which were a high dose of 8 mg/kg and a low dose of 2 mg/kg, respectively, with 17 animals in each group. Dosing was conducted daily until dissection.

(1) The calculation formula of a DAI score: DAI value=(score of weight loss+score of stool trait+score of blood in stool)/3.

A specific scoring method was as shown in the table.

TABLE 1
Scoring Method of DAI

| | Weight | | |
|---|---|---|---|
| Score | Loss % | Stool Trait | Blood in stool |
| 0 | None | Normal | Occult Blood (−) |
| 1 | 1-5 | Soft Stool | Very blurry blue, not |
| | | | easy to distinguish |
| 2 | 5-10 | Fecal wetness and softness | Blurry blue, can |
| | | are very obvious | be distinguished |
| 3 | 10-20 | Semi loose stool | Obvious blue |
| 4 | >20 | Loose stool | Darker and wider |
| | | | range of blue |

(2) Main Organ Coefficient and Organ Preservation

The animals were anesthetized and harvested, and the liver, spleen, thymus and testis were weighed to calculate the organ coefficient; the colon tissue was taken, measured with a ruler for the length, and measured with a vernier caliper for the thickness, and the number and size of tumors and degree of colonic ulceration and adhesion were recorded; respective tissues were cut into small pieces and fixed, and the remaining tissues were frozen and stored; and the fixed tissues were used for HE staining and immunohistochemistry.

(3) Determination of Intestinal Permeability in Serum

On the first day when the modeling is completed and water drinking is recovered in the third stage, 6-8 animals in each group were fasted but given access to water for 12 h, and fed with 40 mg/100 g of FITC-dextran through a gastric tube. After 4 h, blood was taken from the animals, anticoagulated with heparin, and centrifuged at 3500 r/min for 15 min. 25 μl of serum was taken and added with 100 μl PBS, and determined on a microplate reader for its fluorescence intensity, where the wavelength of the exciting light was 480 nm, and the wavelength of the emitting light was 520 nm. A standard curve was made and the sample concentration was calculated.

(4) Determination of Cytokines in Intestinal Tumor Tissues

The intestinal tumor tissues frozen and stored at −80° C. were taken and homogenized, and the homogenate was determined with a BCA kit for the total protein concentration thereof. The homogenate was diluted by 20 times and then determined by Elisa method for cytokines: IL-6, IL-10, and IL-1β. The protein was corrected, and a standard curve is made to calculate the sample concentration.

(5) Typing of Peripheral Blood Macrophages

Peripheral blood was isolated using a lymphocyte separation solution to obtain mononuclear cells, and added with antibodies CD11b, CD86, and CD163 separately according to the instructions.

(6) Determination of Biochemical Indexes in Homogenate of Serum and Intestinal Tissues

After anesthesia and blood sampling, the blood were centrifuged at 3500 r/min for 15 min, and the supernatant was taken to determine the contents of 1,25-(OH)₂D₃ and PGE2 according to the instructions. The intestinal tumor tissues frozen and stored at −80° C. were taken and homogenized to determine the content of PGE2. The protein was corrected, and a standard curve is made to calculate the sample concentration.

(7) Detection of Acting Target of Monotropein

A potential acting target of monotropein for interfering with the colon cancer was detected.

Experimental Results:

(1) Comparison of Body Weight and Colonic Pathological Conditions for Each Group of Animals

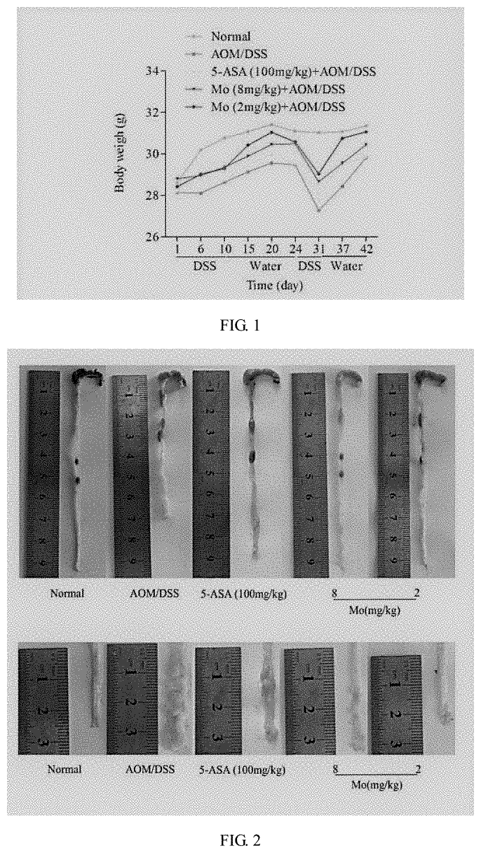

After AOM modeling, the weight loss of the animals was relatively more obvious, individual animals showed loose hair, and the status of the animals was worse than that of the normal group. After 5 days of DSS dosing, some animals of the model group showed symptoms of loose stools and blood in the stool, and slowly recovered body weights. Most of the groups showed a downtrend, and the animals were in poor spirit and had loose and matte hair. After 6 days, most animals developed symptoms of bloody stools, and the body weights of the animals were decreased significantly. After the water was changed back to normal drinking water, the symptoms were gradually disappeared and the body weights rose. An anal prolapse phenomenon occurred in some animals of the model group and the positive group at the second stage of modeling, which was relieved after the water was changed back to conventional drinking water. At the third stage, the anal prolapse occurred in 8-12 animals of each of the model group and the positive group, and the anal prolapse occurred in 2-6 animals in each of the monotropein administration groups. Dissection of mice with anal prolapse showed that, the tumor at the end of the intestine all grew annularly and caused colonic obstruction. Compared with the model group, the administration groups all had different degrees of improvement. The experimental results were shown in FIGS. 1 and 2.

(2) Mouse Organ Coefficient and Intestinal Index

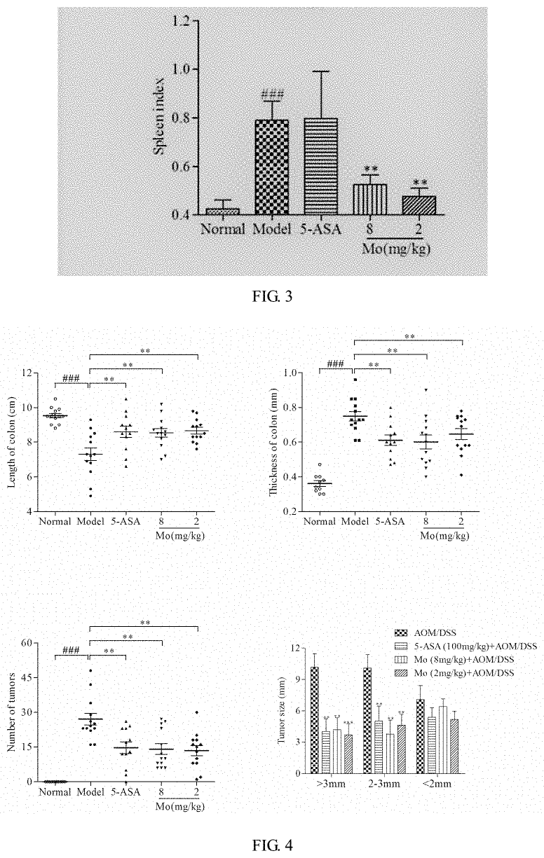

From the organ coefficient, it could be seen that the monotropein groups at high and low doses both can significantly reduce the spleen enlargement caused by AOM/DSS modeling, while 5-ASA has no such effect, suggesting that monotropein may have a certain immune regulation role (the results were shown in FIG. 3). The length and thickness of the colon in the model group were down-regulated and up-regulated respectively as compared with those of the normal group, with significant differences in both the length and thickness of the colon, while each of the three administration groups had different degrees of improvement, with the differences being statistically significant. The number of tumors in each of the three administration groups was significantly reduced at different degrees, and the difference was statistically significant. Additionally, most of the tumors of the model group were large (&gt; 3 mm), while the tumors of the administration groups were relatively small (&lt; 2 mm). The experimental results were shown in FIG. 4.

(3) Detection of Intestinal Permeability in Mice

In order to determine the difference in intestinal permeability of each group of mice, after ending of the third stage of modeling, the intestinal permeability of each group of mice was determined with a microplate reader by using FITC-dextran. It could be seen from the results that, the intestinal permeability of the animals in the model group was significantly increased, and the intestinal permeability in the three administration groups were down-regulated at different degrees, where there were significant differences in the intestinal permeability for the high and low doses of the monotropein, while the difference for the positive administration group was not statistically significant. The experimental results were shown in FIG. 5.

(4) Determination of Cytokine Content in Mouse Tumor Tissues

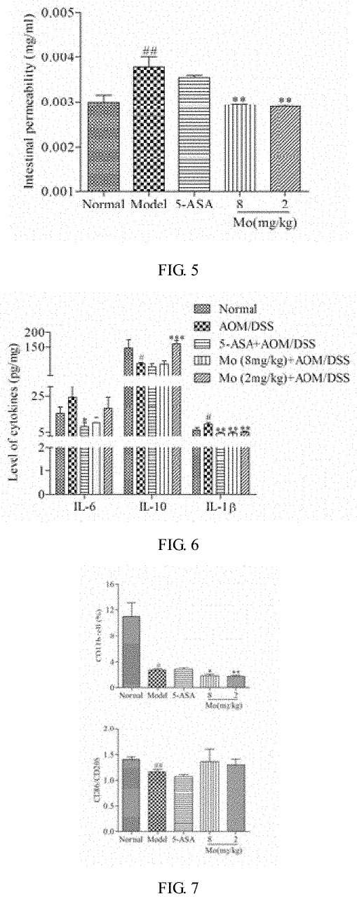

To determine whether periodical gavage of monotropein could change the cytokine levels in intestinal tissues of mice, we determined the levels of IL-6, IL-10 and IL-1β in the tumor tissues by using the Elisa method. The results showed that the contents of IL-6 and IL-1β in the model group were significantly higher than those in the normal group, and were reduced at different degrees in respective administration groups. Furthermore, the level of IL-10 in the model group was significantly reduced as compared with that in the normal group, while there were no significant differences in IL-10 for the positive administration group and the high-dose monotropein group, and the level of IL-10 in the low-dose monotropein group was extremely significantly up-regulated as compared with that of the model group. The experimental results were shown in FIG. 6.

(5) Typing of Mouse Peripheral Blood Macrophages

In order to determine the difference in macrophage polarization in each group of mice, in this experiment the typing of macrophages in peripheral blood of each group of mice was determined by flow cytometry. It could be seen from the results that, the ratio of macrophage in each of the model group and the administration groups was reduced as compared with that in the normal group, the M1/M2 ratio in each of the model group and the positive administration group was also significantly reduced as compared with that in the normal group, and the M1/M2 ratio in the monotropein group has an up-regulating trend as compared with that in the normal group. The test results were shown in FIG. 7.

(6) Determination of Contents of 1,25(OH)₂D₃ and PGE2 in Mice

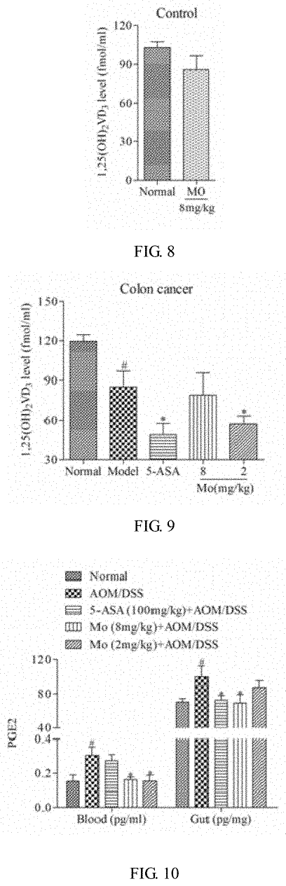

In order to determine whether periodical gavage of monotropein could increase the serum level of 1,25(OH)₂D₃ of mice, the serum concentration of 1,25(OH)₂D₃ was detected in the mice of the blank control group and respective intervention group as well as the normal animal+monotropein group. The results of the experiment showed that, after the administration of the monotropein, the level of 1,25(OH)₂D₃ was down-regulated in the animals of the blank group, but there was no statistical difference. In an AOM/DSS colon cancer model, the level of 1,25(OH)₂D₃ in the model group was significantly reduced as compared with that in the normal group, and the level of 1,25(OH)₂D₃ in each of the administration groups was down-regulated as compared with that in the model group. Therefore, it is speculated that the monotropein did not play its role by promoting up-regulation of 1,25(OH)₂D₃. In order to clarify the effect of the monotropein on inflammatory mediators in the colon cancer model, we determined the content of PGE2 in serum and intestine of mice by the Elisa method. As shown in the drawings, the content of PGE2 in the serum and intestine of the AOM/DSS group were significantly up-regulated as compared with that of the normal group, and the content of PGE2 in the serum and intestine of each of the administration groups were down-regulated at different degrees. The experimental results were shown in FIGS. 8, 9 and 10.

(7) Study of Acting Target of Monotropein:

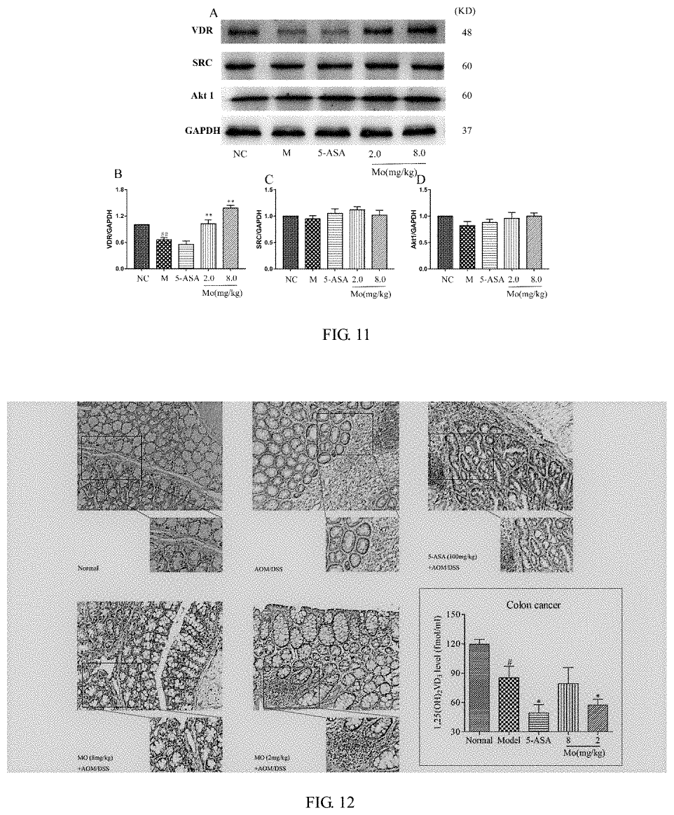

Western blot results showed that the monotropein mainly promoted the expression of intestinal VDR, and had no significant effect on SRC and Aktl. Immunohistochemistry results showed that the monotropein up-regulated the expression of VDR, and the expression of this protein was not related to the secretion of the endogenous hormone 1,25(OH)₂D₃. The experimental results were shown in FIGS. 11 and 12.

Experiment 2: Experimental Study on the Anti-Colon Cancer Effect of Monotropein, Desacetyl Asperulosidic Acid, Asperuloside, and Asperulosidic Acid

Experimental method: 70 Balb/c mice of 6-8 weeks were purchased from the Experimental Animal Center in Guangzhou University of Chinese Medicine, weighed about 18-22 g, and subjected to adaptive feeding for 10 days. Modeling was divided into three stages. In the first stage, except for the normal group of animals being given saline injection, each of the other animals were intraperitoneally injected with a carcinogen AOM (10 mg/kg), and given free access to 2% DSS water from the next day for 7 days, and then given access to replaced regular drinking water for 14 days. In the second stage, the animals were given free access to 2% DSS water for 10 days, and then given access to replaced regular drinking water for 14 days. In the third stage, the animals were given free access to 2% DSS water for 10 days, and then given access to replaced regular drinking water for 11 days. The normal group was given access to regular drinking water throughout the experiment. The diet, water drinking and body weights of the animals were recorded daily and DAI scores were made, and the animals were killed and harvested at the 10th week. In this experiment, the animals were divided into groups and administrated on the first day of a modeling period of the second stage, where a total of 7 groups were set, each group including 10 animals. The first group was a blank control group (control) that was given access to drinking water by gavage; the second group was a model group (model), in which a total of 10 animals were given access to drinking water by gavage; the third group was a vitamin D3 positive administration control group (VD3, 0.2 mg/kg); and the fourth to seventh groups were given monotropein, desacetyl asperulosidic acid, asperuloside, and asperulosidic acid (groups labeled as: Mo, As, Aa, Da), respectively at a given dose of 2 mg/kg. Dosing was conducted once a day until the end of the experiment. At the end of the experiment, the animals were anesthetized and harvested, and the colon tissue was taken, measured with a ruler for the length, and measured with a vernier caliper for the thickness. The effects of monotropein, desacetyl asperulosidic acid, asperuloside and asperulosidic acid on expression of the vitamin D receptor were detected.

Experimental Results:

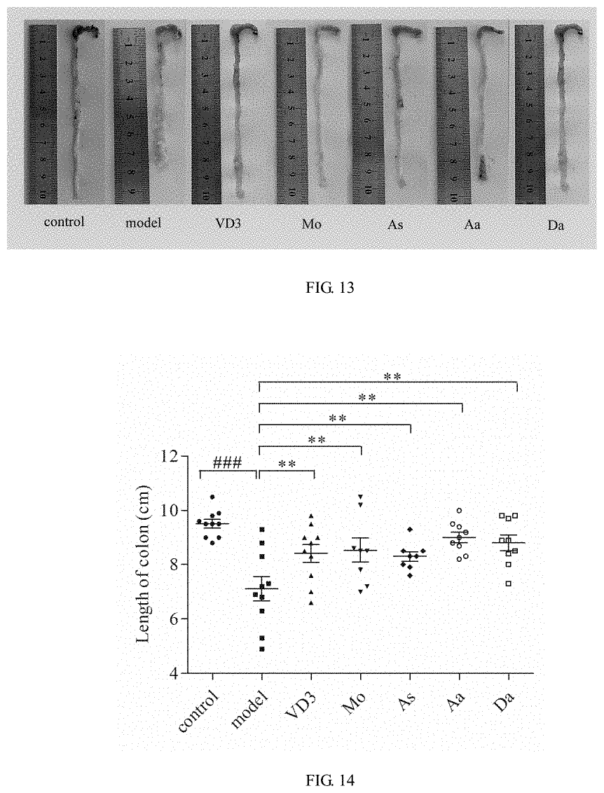

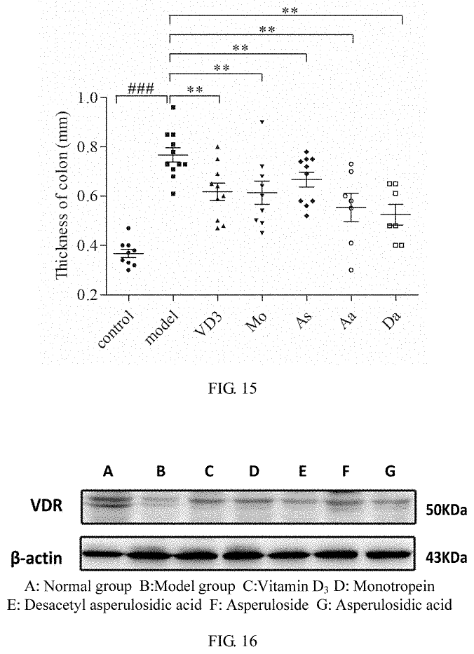

Compared with the normal group, the length and thickness of the colon in the model group were down-regulated and up-regulated respectively as compared with those of the normal group, and in the model group confluent tumors were grown at the colon end, while the five administration groups showed different degrees of significant improvement. The experimental results were shown in FIGS. 13, 14 and 15. The experimental results showed that each of the monotropein, desacetyl asperulosidic acid, asperuloside, and asperulosidic acid had an effect of inhibiting the incidence and development of colorectal cancers.

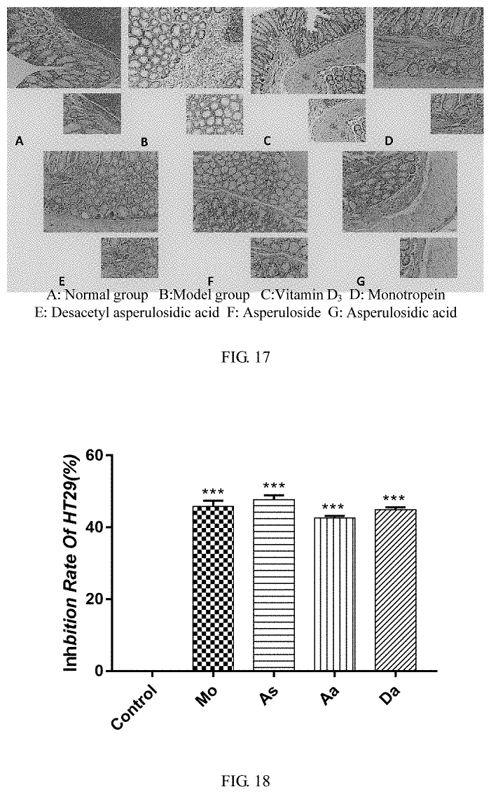

Both Western blot results and immunohistochemical results showed that: as compared with the blank group, each of vitamin D3, monotropein, desacetyl asperulosidic acid, asperuloside, and asperulosidic acid could promote expression enhancement of the vitamin D receptor in the colon tissues. The experimental results were shown in FIGS. 16 and 17.

Experiment 3: Experimental Study on the Inhibition Effect of the Monotropein, Desacetyl Asperulosidic Acid, Asperuloside, and Asperulosidic Acid on Proliferation of Colon Cancer Cells, Breast Cancer Cells and Pancreatic Cancer Cells

1. Experiment of Transplanted Tumors of HT29 Colon Cancer Cells

(1) Culturing of HT29 Colon Cancer Cells

The HT29 cells were thawed, and cultured in a changed medium after 24 hours. Medium preparation method: 6% serum, 1% of a bispecific antibody+a 1640 basal medium. After grown to confluent, the cells were digested and subcultured to expand the number of cells. When reached the required amount, the cells were digested by trypsin, centrifuged, and resuspended in PBS to prepare a cell suspension of 1×10⁸ cells/ml. A part of the cell suspension was added with the monotropein, desacetyl asperulosidic acid, asperuloside, and asperulosidic acid respectively, and the inhibition rate (%) of tumor cell proliferation by each of these different compounds was examined.

(2) Feeding and Grouping of Nude Mice

Male adult nude mice, which had the body weights of 20-24 g, were purchased from Guangdong Medical Laboratory Animal Center. After 7 days of adaptive feeding of the animals, except for the blank group, the remaining animals were injected with 0.2 ml of a cell suspension in PBS at ventrodorsal and scapula sites of them, the response conditions of the animals were observed, and the body weights of the animals were determined once a week. After 2 weeks of observation, the animals were grouped into the following groups according to tumor size: a blank group; a model group; a 5-fluorouracil control group (5-Full, 40 mg/kg); monotropein+5-fluorouracil (2 mg/kg monotropein+20 mg/kg 5-fluorouracil), desacetyl asperulosidic acid+5-fluorouracil (2 mg/kg desacetyl asperulosidic acid+20 mg/kg 5-fluorouracil), asperuloside+5-fluorouracil (2 mg/kg asperuloside+20 mg/kg 5-fluorouracil), asperulosidic acid+5-fluorouracil (2 mg/kg asperulosidic acid+20 mg/kg 5-fluorouracil), these groups being labeled as: Mo, As, Aa, Da. Each group included 8 animals; and administration was conducted once a day.

(3) Animal-Related Observation and Determination of Tumor Size

The status, death conditions, and eating and drinking conditions of the animals were observed daily. The body weights of the animals were determined once a week and the tumor size of the animals was determined every 4 days. The tumor volume of the animals was determined by using a vernier caliper. After 1 month of administration, the animals were sacrificed after blood was sampled from the orbit thereof, and the tumor was separated and weighed.

2. Experiment of Transplanted Tumors of SW1990 Breast Cancer Cells

(1) Culturing of SW1990 Breast Cancer Cells

The SW1990 cells were thawed, and cultured in a changed medium after 24 hours. Medium preparation method: 6% serum, 1% of a bispecific antibody+a 1640 basal medium. After grown to confluent, the cells were digested and subcultured to expand the number of cells. When reached the required amount, the cells were digested by trypsin, centrifuged, and resuspended in PBS to prepare a cell suspension of 1×10⁸ cells/ml. A part of the cell suspension was added with the monotropein, desacetyl asperulosidic acid, asperuloside, and asperulosidic acid respectively, and the inhibition rate (%) of tumor cell proliferation by each of these different compounds was examined.

(2) Feeding and Grouping of Nude Mice

Male adult nude mice, which had the body weights of 20-24 g, were purchased from Guangdong Medical Laboratory Animal Center. After 7 days of adaptive feeding of the animals, except for the blank group, the remaining animals were injected with 0.2 ml of a SW1990 cell suspension in PBS at ventrodorsal and scapula sites of them, the response conditions of the animals were observed, and the body weights of the animals were determined once a week. After 2 weeks of observation, the animals were grouped into the following groups according to tumor size: a blank group; a model group; a 5-fluorouracil control group (5-Full, 40 mg/kg); monotropein+5-fluorouracil (2 mg/kg monotropein+20 mg/kg 5-fluorouracil), desacetyl asperulosidic acid+5-fluorouracil (2 mg/kg desacetyl asperulosidic acid+20 mg/kg 5-fluorouracil), asperuloside+5-fluorouracil (2 mg/kg asperuloside+20 mg/kg 5-fluorouracil), asperulosidic acid+5-fluorouracil (2 mg/kg asperulosidic acid+20 mg/kg 5-fluorouracil), these groups being labeled as: Mo, As, Aa, Da. Each group included 8 animals; and administration was conducted once a day.

(3) Animal-Related Observation and Determination of Tumor Size

The status, death conditions, and eating and drinking conditions of the animals were observed daily. The body weights of the animals were determined once a week and the tumor size of the animals was determined every 4 days. The tumor volume of the animals was determined by using a vernier caliper. After 1 month of administration, the animals were sacrificed after blood was sampled from the orbit thereof, and the tumor was separated and weighed.

3. Experiment of Transplanted Tumors of MB-231 Pancreatic Cancer Cells

(1) Culturing of SMB-231 Pancreatic Cancer Cells

The SMB-231 pancreatic cancer cells were thawed, and cultured in a changed medium after 24 hours. Medium preparation method: 6% serum, 1% of a bispecific antibody+a 1640 basal medium. After grown to confluent, the cells were digested and subcultured to expand the number of cells. When reached the required amount, the cells were digested by trypsin, centrifuged, and resuspended in PBS to prepare a cell suspension of 1×10⁸ cells/ml. A part of the cell suspension was added with the monotropein, desacetyl asperulosidic acid, asperuloside, and asperulosidic acid respectively, and the inhibition rate (%) of tumor cell proliferation by each of these different compounds was examined.

(2) Feeding and Grouping of Nude Mice

Male adult nude mice, which had the body weights of 20-24 g, were purchased from Guangdong Medical Laboratory Animal Center. After 7 days of adaptive feeding of the animals, except for the blank group, the remaining animals were injected with 0.2 ml of a SMB-231 pancreatic cancer cell suspension in PBS at ventrodorsal and scapula sites of them, the response conditions of the animals were observed, and the body weights of the animals were determined once a week. After 2 weeks of observation, the animals were grouped into the following groups according to tumor size: a blank group; a model group; a 5-fluorouracil control group (5-Full, 40 mg/kg); monotropein+5-fluorouracil (2 mg/kg monotropein+20 mg/kg 5-fluorouracil), desacetyl asperulosidic acid+5-fluorouracil (2 mg/kg desacetyl asperulosidic acid+20 mg/kg 5-fluorouracil), asperuloside+5-fluorouracil (2 mg/kg asperuloside+20 mg/kg 5-fluorouracil), asperulosidic acid+5-fluorouracil (2 mg/kg asperulosidic acid+20 mg/kg 5-fluorouracil), these groups being labeled as: Mo, As, Aa, Da. Each group included 8 animals; and administration was conducted once a day.

(3) Animal-Related Observation and Determination of Tumor Size

The status, death conditions, and eating and drinking conditions of the animals were observed daily. The body weights of the animals were determined once a week and the tumor size of the animals was determined every 4 days. The tumor volume of the animals was determined by using a vernier caliper. After 1 month of administration, the animals were sacrificed after blood was sampled from the orbit thereof, and the tumor was separated and weighed.

Experimental Results:

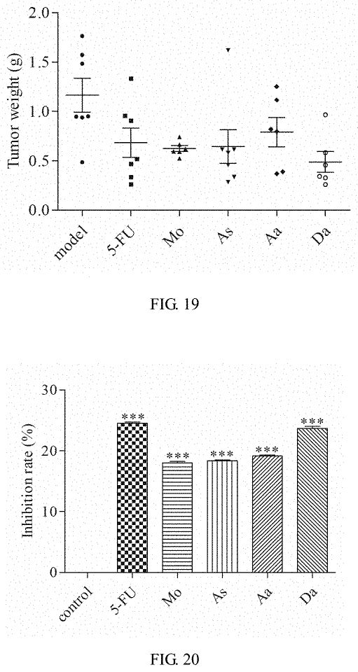

(1) The results of the in vitro cell proliferation inhibition experiment showed that: as compared with the blank control group, each of monotropein, desacetyl asperulosidic acid, asperuloside, and asperulosidic acid could significantly inhibit the proliferation of HT29 colorectal cancer cells at a concentration of 1 uM (see FIG. 18). In a model of tumor-bearing mice, administration of a 5-fluorouracil chemotherapeutic drug at a half dose (20 mg/kg) in combination with the monotropein, desacetyl asperulosidic acid, asperuloside, and asperulosidic acid respectively, could significantly inhibit tumor proliferation, with the effect being comparable to that of the group of chemotherapy alone (the dose of 5-fluorouracil is 40 mg/kg). It is suggested that, the combined administration of the monotropein, desacetyl asperulosidic acid, asperuloside, and asperulosidic acid with the chemotherapeutic drug, could reduce the dosage of the chemotherapeutic drug and had the effect of inhibiting the proliferation of HT29 colorectal cancer cells (see FIG. 19).

(2) The results of the in vitro cell proliferation inhibition experiment showed that: as compared with the blank control group, each of monotropein, desacetyl asperulosidic acid, asperuloside, and asperulosidic acid could significantly inhibit the proliferation of HT29 colorectal cancer cells at a concentration of 1 uM (see FIG. 20). In a model of tumor-bearing mice, administration of a 5-fluorouracil chemotherapeutic drug at a half dose (20 mg/kg) in combination with the monotropein, desacetyl asperulosidic acid, asperuloside, and asperulosidic acid respectively, could significantly inhibit tumor proliferation, with the effect being comparable to that of the group of chemotherapy alone (the dose of 5-fluorouracil is 40 mg/kg). It is suggested that, the combined administration of the monotropein, desacetyl asperulosidic acid, asperuloside, and asperulosidic acid with the chemotherapeutic drug, could reduce the dosage of the chemotherapeutic drug and had the effect of inhibiting the proliferation of SW1990 breast cancer cells (see FIG. 21).

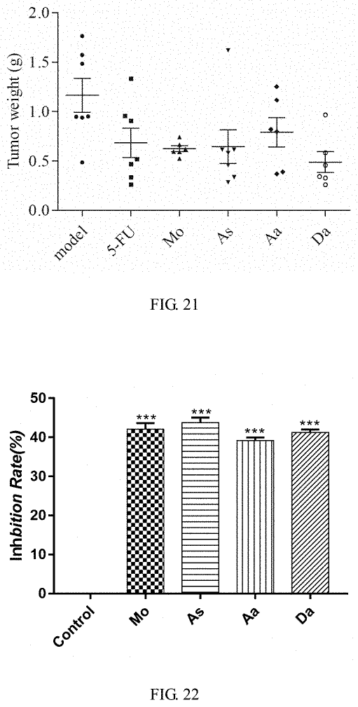

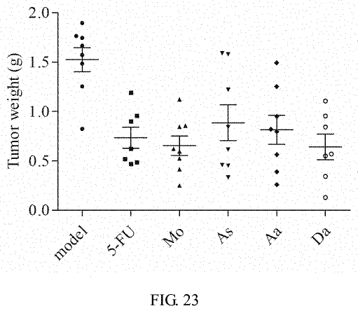

(3) The results of the in vitro cell proliferation inhibition experiment showed that: as compared with the blank control group, each of monotropein, desacetyl asperulosidic acid, asperuloside, and asperulosidic acid could significantly inhibit the proliferation of SMB-231 pancreatic cancer cells at a concentration of 1 uM (see FIG. 22). In a model of tumor-bearing mice, administration of a 5-fluorouracil chemotherapeutic drug at a half dose (20 mg/kg) in combination with the monotropein, desacetyl asperulosidic acid, asperuloside, and asperulosidic acid respectively, could significantly inhibit tumor proliferation, with the effect being comparable to that of the group of chemotherapy alone (the dose of 5-fluorouracil is 40 mg/kg). It is suggested that, the combined administration of the monotropein, desacetyl asperulosidic acid, asperuloside, and asperulosidic acid with the chemotherapeutic drug, could reduce the dosage of the chemotherapeutic drug and had the effect of inhibiting the proliferation of SMB-231

What is claimed is:

1. A vitamin D receptor agonist, comprising one or more of monotropein, desacetyl asperulosidic acid, asperuloside, and asperulosidic acid.

2. Use of the vitamin D receptor agonist according to claim 1 in preparation of a medicament for the treatment and/or prevention of a cancer.

3. The use according to claim 2, wherein the cancer is a colon cancer.

4. The use according to claim 2, wherein the cancer is a breast cancer.

5. The use according to claim 2, wherein the cancer is a pancreatic cancer.

6. A pharmaceutical preparation prepared from the vitamin D receptor agonist according to claim 1, wherein the pharmaceutical preparation uses one or more of monotropein, desacetyl asperulosidic acid, asperuloside, and asperulosidic acid as active ingredients.

7. The pharmaceutical preparation according to claim 6, wherein the pharmaceutical preparation comprises an oral preparation and an injection preparation.

8. Use of one or more of monotropein, desacetyl asperulosidic acid, asperuloside, and asperulosidic acid in preparation of a medicament for the treatment and/or prevention of a cancer after the activation of the vitamin D receptors.

9. Use of the vitamin D receptor agonist according to claim 1 for the treatment and/or prevention of a cancer.

10. The use according to claim 9, wherein the cancer comprises the colon cancer, the breast cancer and the pancreatic cancer.
